(19) United States
(12) Patent Application Publication
(10) Pub. No.: US 2006/0246036 A1
(43) Pub. Date: Nov. 2, 2006

(54) Delivery of therapeutics to the brain and spinal cord

(75) Inventors: Jonathan W. Francis, Walpole, MA (US); Robert H. Brown Jr, Needham, MA (US); Paul S. Fishman, Baltimore, MD (US)

Correspondence Address: WOLF GREENFIELD & SACKS, PC, FEDERAL RESERVE PLAZA, 600 ATLANTIC AVENUE, BOSTON, MA, 02210-2206, US

(73) Assignees: The General Hospital Corporation, Boston, MA (US); University of Maryland, Baltimore, Baltimore, MD (US)

(21) Appl. No.: 10/526,579

(22) PCT Filed: Sep. 4, 2003
(86) PCT No.: PCT/US03/27816

§ 371 Date: Jan. 9, 2006

Related U.S. Application Data
(60) Provisional application No. 60/408,577, filed on Sep. 6, 2002.

(51) Int. Cl. A61K 48/00; A61K 39/08

(52) U.S. Cl. 424/93.2; 424/239.1; 514/44

(57) ABSTRACT

The invention provides compositions and methods for the delivery of therapeutic compounds to the brain and spinal cord.

### GOVERNMENT SUPPORT

This work was funded in part by grant number NS38679 from the National Institutes of Health. Accordingly, the United States Government may have certain rights in this invention.

### FIELD OF THE INVENTION

This invention relates to compositions and methods for the delivery of therapeutic compounds to the brain and spinal cord.

### BACKGROUND OF THE INVENTION

Tetanus toxin, when administered systemically or intramuscularly to animals, is selectively taken up by motor neurons in the brainstem and spinal cord. Tetanus toxin has a dichain structure in which the heavy chain appears to mediate binding, and the light chain is responsible for most of the toxicity. The carboxyl 451 amino acid fragment of the heavy chain (“tetanus toxin fragment C” or “TTC”) retains the neuronal binding and uptake properties of the holotoxin but the TTC fragment lacks the toxic domains of the holotoxin (Bizzini et al., J. Neurochem., 28: 529-542, 1977; Morris, et al., J. Biol. Chem., 255:6071-6076, 1980; Weller et al., Toxicon, 24: 1055-1063, 1986). TTC has been chemically conjugated to large proteins to enhance their uptake into neurons in tissue culture (Dobrenis et al., Proc. Natl. Acad. Sci. USA, 89:2297-2301, 1992) and neurons in animal models (Bizzini et al., Brain Res., 193:221-227, 1980; Beaude et al., Biochem. J., 271: 87-91, 1980; Fishman et al., J. Neurol. Sci., 98: 311-325, 1990).

The ability of a hybrid protein comprised of human superoxide dismutase (hSOD-1) monomer fused at its carboxyl terminus to the amino terminus of TTC to be selectively taken up by neurons in a dose-dependent manner was disclosed in U.S. Pat. No. 5,780,024. The hybrid protein, generally known as SOD:Tet451 or SOD:TTC, retained superoxide dismutase enzymatic activity after neuronal uptake and was shown to undergo retrograde axonal transport in motor neurons in vivo. Transport across brain synapses (“transsynaptic transport”) of TTC and TTC fusion proteins has been shown to provide for migration of a TTC fusion proteins from motor neurons into second order neurons of the brain (e.g., Fishman and Savitt, Exp. Neurol. 106(2):197-203, 1989; Figueiredo et al., Exp. Neurol. 145:546-554, 1997; Coen et al., Proc. Natl. Acad. Sci. USA 94:9400-9405, 1997; Schwab et al., J. Cell Biol. 82:798-810,1979).

Thus, the use of TTC as a vehicle to deliver therapeutic proteins to motor neurons via retrograde axonal transport from the periphery is well established. Such delivery can be accomplished after intramuscular injection, but has been observed following systemic administration as well (intraperitoneal, intravenous). Such delivery targets motor neurons and selected other types of neurons such as preganglionic sympathetic neurons, and transfer of the therapeutic protein to a limited number of neurons with direct synaptic contact to targeted motor neurons has been described. The distribution of this so-called transneuronal or transsynaptic label has been described by Coen et al. as discrete; it is discrete because it is localized to a very small number of “second order” CNS neurons that have direct synaptic contact to the “first order” motor neurons. Thus, in the report by Coen et al., because tongue muscles were injected with TTC, anti-TTC immunostaining was largely restricted to the hypoglossal nucleus in the brainstem (which contains the cell bodies of the motor neurons innervating the tongue muscles) and other brainstem neurons known to have synaptic inputs to the hypoglossal nucleus.

While TTC is useful for delivery of proteins to the central nervous system via retrograde transport from the periphery and transsynaptic transport, this route of uptake allows a passenger protein (i.e., a protein linked to TTC) to access only a small fraction of neurons in the CNS, namely motor neurons in the spinal cord and brainstem and neurons with direct synaptic contact to these motor neurons. Unfortunately, many if not most neurological diseases that would benefit from the neuron binding and internalization properties of TTC affect neurons in other regions of the brain and spinal cord that are inaccessible to TTC taken up by retrograde transport from the periphery. These other regions include virtually all of the structures in the brain, brainstem and cerebellum such as the cerebral and cerebellar cortices as well as multiple neuronal nuclei situated deeply within the brain, brainstem and cerebellum. These other regions also include the dorsal horn neurons in the spinal cord. Existing methods for delivery of proteins to the CNS using TTC do not provide adequate penetration to these regions of the brain and spinal cord.

### SUMMARY OF THE INVENTION

It has now been discovered that tetanus toxin C fragment can be used to deliver therapeutic proteins to regions of the brain and spinal cord not accessible to TTC following peripheral (intramuscular) administration and subsequent retrograde transport and transsynaptic transport. When delivered to the brain by direct injection into the brain or by infusion into cerebrospinal fluid, hybrid proteins comprised of TTC and a therapeutic protein unexpectedly diffuse widely throughout the brain and spinal cord, in contrast to the point-to-point distribution that would be expected following retrograde transport and transsynaptic transport from intramuscular injection.

According to one aspect of the invention, methods for administering a therapeutic molecule to a subject are provided. The methods include providing a hybrid protein comprising the therapeutic molecule and tetanus toxin fragment C, and administering the hybrid protein by infusion of the hybrid protein into the cerebrospinal fluid.

According to another aspect of the invention, methods for administering a therapeutic molecule to a subject are provided. The methods include providing a hybrid protein comprising the therapeutic molecule and tetanus toxin fragment C, and administering the hybrid protein directly into the brain or spinal cord parenchyma.

According to a further aspect of the invention, methods for administering a therapeutic molecule to a region of a subject's brain and spinal cord that is not accessible via retrograde transport or transsynaptic transport from motor neurons are provided. The methods include providing a hybrid protein comprising the therapeutic molecule and tetanus toxin fragment C, and administering the hybrid protein by infusion of the hybrid protein into the cerebrospinal fluid.

According to still another aspect of the invention, methods for administering a therapeutic molecule to a region of a subject's brain and spinal cord that is not accessible via retrograde transport or transsynaptic transport from motor neurons are provided. The methods include providing a hybrid protein comprising the therapeutic molecule and tetanus toxin fragment C, and administering the hybrid protein directly into the brain or spinal cord parenchyma.

According to yet another aspect of the invention, methods for treating a neurological disorder are provided. The methods include administering to a subject in need of such treatment an effective amount of a hybrid protein comprising tetanus toxin fragment C and a therapeutic molecule by infusion of the hybrid protein into the cerebrospinal fluid.

According to a further aspect of the invention, methods for treating a neurological disorder are provided. The methods include administering to a subject in need of such treatment an effective amount of a hybrid protein comprising tetanus toxin fragment C and a therapeutic molecule by administering the hybrid protein directly into the brain or spinal cord parenchyma.

In certain embodiments of the foregoing methods, the subject has a neurological disorder selected from the group consisting of cerebrovascular accidents (stroke), amyotrophic lateral sclerosis, Parkinson's disease, Huntington's disease, Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, striatonigral degeneration, torsion dystonia, familial tremor, Down's Syndrome, Gilles de la Tourette syndrome, Hallervorden-Spatz disease, peripheral neuropathies, dementia pugilistica, AIDS dementia, age-elated dementia, age-associated memory impairment, amyloidosis-related neurodegenerative diseases, traumatic brain and spinal cord injury, cerebral edema, schizophrenia, peripheral nerve damage, spinal cord injury, and Wernicke-Korsakoff's related dementia.

In some embodiments of the invention, the therapeutic molecule is a protein or peptide. In such embodiments, the protein preferably is selected from the group consisting of GDNF, BDNF, LIF, cardiotrophin (CT-1), FGF, HGF, insulin-like growth factors 1 and 2 (IGF-1, IGF-2) and the neurotrophins. In other embodiments of the invention, the therapeutic molecule is a nucleic acid molecule, a virus, an antibody or fragment thereof, a lipid, a polysaccharide, an oligonucleotide or a modified or derivatized oligonucleotide, an RNA molecule or a modified or derivatized oligoribonucleotide, a plasmid, cosmid, bacmid or vehicle for the packaging and/or expression of clonal DNA and/or a ribozyme.

In certain embodiments of the invention, the mode of administration is intracerebroventricular administration. In other embodiments, the mode of administration is intrathecal infusion. For these modes of administration, it is preferred that the hybrid protein is administered using a pump.

In some embodiments of the invention, the hybrid protein is administered by injection or by infusion.

When infused, it is preferred that the hybrid protein is infused for 1 or more days, more preferably for 3 or more days, still more preferably for 1 or more weeks.

In the foregoing methods, it is preferred that the hybrid protein is administered to at least about 10% of brain volume. More preferably, the hybrid protein is administered to at least about 30% of brain volume. Still more preferably, the hybrid protein is administered to at least about 50% of brain volume.

In a further aspect of the invention, the foregoing methods utilize intranasal administration.

Medical devices and pharmaceutical formulations for performing the foregoing methods also are provided, as are the use of the disclosed hybrid proteins comprising tetanus toxin fragment C and a therapeutic molecule in the preparation of medicaments, particularly medicaments for treatment of the diseases listed herein.

In a manner similar to that described for the delivery of therapeutic molecules herein, TTC can be used to deliver diagnostic molecules to the brain and spinal cord as well.

These and other aspects of the invention, as well as various advantages and utilities, will be more apparent with reference to the detailed description of the preferred embodiments.

### BRIEF DESCRIPTION OF THE DRAWINGS

FIGS. 1 and 2 respectively depict images of coronal sections of whole rat brains showing the distribution of anti-TTC (FIG. 1) and anti-SOD-1 (FIG. 2) immunostaining following intraventricular infusions of phosphate-buffered saline (PBS), hSOD-1, TTC or SOD:TTC.

FIGS. 3 and 4 respectively depict images of cross sections of rat cervical spinal cord showing the distribution of anti-TTC (FIG. 3) and anti-SOD-1 (FIG. 4) immunostaining following intraventricular infusions of PBS, hSOD-1, TTC or SOD:TTC.

FIG. 5 shows the distribution of anti-TTC and anti-SOD-1 immunostaining in coronal sections of whole mouse brain following intraparenchymal injection of hSOD-1, TTC or SOD:TTC. Details are provided in the text below.

### DETAILED DESCRIPTION OF THE INVENTION

The invention provides methods for efficient and widespread delivery of therapeutic proteins to the brain and spinal cord. Such methods are useful in treating a variety of conditions in which delivery of therapeutic proteins to the brain and spinal cord is desirable, including but not limited to neurodegenerative disorders, cerebrovascular accidents (stroke) and traumatic injury to the brain and spinal cord.

We have discovered that a hybrid protein composed of TTC and a therapeutic protein, when administered by direct injection into the brain or continuous infusion into cerebrospinal fluid, provides widespread delivery of the therapeutic protein to many areas of the brain and spinal cord that would otherwise not be accessible to the therapeutic protein itself following administration by these routes.

There are no previous reports or published hypotheses concerning the use of TTC as a vector to facilitate the delivery of therapeutic proteins via direct intracerebral injection or infusion into the cerebrospinal fluid of cerebral ventricles, CNS cisterns or subarachnoid spaces surrounding the brain and spinal cord. Generalization from the distribution of TTC and TTC linked proteins described by Coen et al. suggests that TTC would not be useful via these routes. Published studies would predict that the only neurons capable of binding and/or taling up TTC following direct intraparenchymal injection would be those “first-order” cells having direct contact with the injection site and a second population of neurons that have direct synaptic contacts with the first order cells.

Our experiments have demonstrated that the distribution of TTC and TTC linked proteins after intracerebral administration or intraventricular infusion into the cerebrospinal fluid is vastly different from that seen after intramuscular injection. This novel distribution and its implications for delivery of therapeutic proteins to the brain and spinal cord are unexpected in view of the knowledge of distribution of TTC after intramuscular injection as described in previous studies.

As shown in the examples below, intracerebral injection or intraventricular infusion of TTC or TTC hybrid proteins (e.g., SOD:TTC) results in an extensive distribution of the test proteins throughout the brain with a much larger range of distribution and superior retention of these proteins compared to non-TTC linked proteins (SOD, albumin). Discrete patches of neuronal labeling similar to that described by Coen et al. (Proc. Natl. Acad. Sci. USA 94:9400-9405, 1997) were not seen in the present study; rather, TTC and the TTC-hybrid protein were very widely distributed. In the present experiments, TTC and SOD:TTC were localized primarily in the extracellular space and neuropil. In some regions, these proteins appeared to be concentrated in discrete, punctate structures surrounding large neurons. Such structures appear to be presynaptic terminals that are known to be one of the preferential sites of TTC localization in cell culture.

Although not wishing to be bound by a particular mechanism, we propose that TTC and the TTC-linked protein are disseminated throughout the brain by binding to abundant TTC receptors known to be present on all neurons. That is, the TTC and TTC-linked proteins appear to migrate widely throughout the brain from the site of introduction using a process of receptor-mediated diffusion.

Following intracerebroventricular infusion, both TTC and the SOD:TTC fusion protein appeared to be most concentrated in brain areas adjacent to the ventricles. The intensity of immunolabeling gradually decreased with increasing distance from the ventricles. With an infusion of three days duration, the protein is detectable in over 50% of the volume of the brain (for TTC) and approximately 30-50% of brain volume for SOD:TTC. The compartmental localization of TTC and SOD:TTC in the brain after intraventricular infusion is identical to that seen after intracerebral injection, with protein mostly detectable in the extracellular space and structures compatible with synaptic terminals. Once again, discrete patches of labeled neurons suggestive of retrograde axonal transport were not observed; rather, there was widespread distribution in a gradient from the site of introduction of the proteins, with concentration of the TTC and TTC-linked proteins around the edges of neurons where the TTC receptors are located.

TTC infused into the ventricle was also distributed throughout both the dorsal and ventral gray matter of the cervical spinal cord. Within the ventral horn of the spinal cord gray matter, large motor neurons were observed to contain anti-TTC immunoreactive granules in a perinuclear location. Intense labeling of synaptic boutons located on the surface of motor neuron cell bodies was also seen. The distribution of SOD:TTC after intraventricular infusion was similarly concentrated in the superficial portions of the dorsal and ventral horn gray matter and also around the central canal of the spinal cord. Moderate to strong labeling of motor neurons in the ventral horn with apparent intracytoplasmic endosomes was observed. Only this limited aspect of the distribution of SOD:TTC following intracerebroventricular infusion has any resemblance to the localization of SOD:TTC observed after retrograde axonal transport from muscle.

The novel and unpredicted distribution of TTC and SOD :TTC by these previously uninvestigated routes of delivery has direct implications for therapy. The extensive distribution of TTC and TTC fusion proteins in CNS following intraparenchymal injection or intraventicular infusion might thus allow TTC to treat disorders that otherwise would not be significantly impacted by a point-to-point distribution of a therapeutic protein. We believe that TTC will improve delivery to the CNS of not only therapeutic proteins but also a wide range of other therapeutic molecules including but not limited to peptides, antibodies, lipids, polysaccharides, oligonucleotides, RNA molecules, plasmids, ribozymes, viruses and combinations thereof.

Because TTC targets the extracellular space and extracellular surface of neuronal cell membranes, it can be used to enhance the delivery of trophic factors such as nerve growth factor (NGF), brain-derived neurotrophic factor (BDNF), neurotrophin 3 (NT3), glial cell line-derived neurotrophic factor (GDNF), or ciliary neurotrophic factor (CNTF). Other growth factors that may be delivered to the brain and spinal cord include: neurotrophin 4/5 (NT4/5), leukemia inhibitory factor (LIF), cardiotrophin (CT-1), insulin-like growth factors 1 and 2 (IGF-1, IGF-2), transforming growth factor alpha (TGF-alpha), transforming growth factor beta 1-3 (TGF-betal, TGF-beta2, TGF-beta3), neurturin (NTN), artemin (ART), persephin (PSP), acidic fibroblast growth factor (FGF-1), basic fibroblast growth factor (FGF-2), fibroblast growth factor-5 (FGF-5), platelet-derived growth factor (PDGF) and stem cell factor (SCF). Trophic factors such as but not limited to these have potential therapeutic use in many disorders of brain and spinal cord including neurodegenerative diseases, trauma and stroke. The neurodegenerative diseases that are potential targets for these therapies include Alzheimer's disease, Huntington's disease, Parkinson's disease, amyotrophic lateral sclerosis (ALS or Lou Gehrig's disease) and other disorders characterized by degeneration of the brain and spinal cord.

Enzymes that would be useful in the peri-neuronal extracellular space include amyloid degrading enzymes for Alzheimer's Disease (e.g., the neprilysin (NEP) family of zinc metalloproteinases, such as NEP and endothelin-converting enzyme, insulysin, angiotensin-converting enzyme, matrix metalloproteinases, plasmin and thimet oligopeptidase (endopeptidase-24.15)), and glutamate degrading enzymes for treatment of ischemic and traumatic brain injury as well as the neurodegenerative diseases mentioned earlier. TTC could enhance the delivery of antibodies to the peri-neuronal extracellular space to protect neurons from viruses such as HIV, Herpes Simplex, equine encephalitis, West Nile encephalitis, or toxins such as tetanus toxin.

Since it is likely that TTC linked proteins will undergo endocytosis from the extracellular space, TTC would enhance the delivery of lysosomal enzymes for inherited storage diseases such as Tay-Sachs (beta-hexosaminidase A (HexA)), Gaucher (acid beta-D-glucosidase), Niemann-Pick disease (acid sphingomyelinase), and neuronal ceroid lipofuscinoses (palmitoyl-protein thioesterase and tripeptidyl amino peptidase-I).

Neurotransmitter synthesizing enzymes that may be delivered to the extracellular perisynaptic space include choline acetyltransferase (ChAT) for Alzheimer's disease, and glutamic acid decarboxylase (GAD) for epilepsy or Huntington's disease. TTC linked proteins can be produced by recombinant techniques or by chemical and reversible conjugation. TTC linked proteins that include pore forming/translocation domains can be used to deliver other neurotransmitter synthesizing enzymes such as tyrosine hydroxylase (TH) for Parkinson's disease, or dopamine beta-hydroxylase and/or tryptophan hydroxylase for depression.

Targeting of the superficial laminae of the dorsal horn could facilitate the delivery of pain relieving proteins such as endorphins, enkephalins, anti-substance P antibodies, or substance P receptor antagonists. Other therapeutic proteins deliverable by the method of the invention will be known to one of ordinary skill in the art.

A partial list of candidate proteins to be delivered for particular diseases is shown in Table 1.

TABLE 1
Proteins for CNS Delivery

| Protein Category | Disease Targets |
|---|---|
| Growth factors | ALS, AD, PD, CVA, SCI, ONND |
| GDNF | |
| neurturin | |
| artemin | |
| BDNF | |
| LIF | |
| Cardiotrophin | |
| FGF | |
| HGF | |
| Neurotrophins | |
| Anti-oxidants | ALS, AD, PD, CVA, SCI, ONND, |
| SOD1 | Inflammation (e.g. MS) |
| SOD2 | |
| Glutathione peroxidase | |
| Catalase | |
| Anti-apoptotics | ALS, AD, PD, CVA, SCI, ONND, |
| Bcl-2 | Inflammation (e.g. MS) |
| CrmA | |
| Baculoviral IAPs | |
| Mammalian IAPs | |
| Proteosome enhancers | ALS, AD, PD, CVA, SCI, ONND |
| Kinase inhibitors | ALS, AD, PD, CVA, SCI, ONND |
| Cell cycle inhibitors | Anti-cancer Therapy |
| Glutamate transport enhancers | ALS, AD, PD, CVA, SCI, ONND |
| EAAT2/GLT1 | |
| Glutamate metabolizers | |
| Glutamate decarboxylase | |
| Analgesics | Pain syndromes |
| Enkephalins | |
| Endorphins | |
| Substance P antagonists | |
| Anti-protein antibodies | |
| e.g. HIV, HSV | AIDS, HIV |
| beta-amyloid protein | AD |
| Lysosomal enzymes | Lysosomal storage diseases |
| Neurotransmitter synthesizing | |
| enzymes | |
| GAD | Epilepsy |
| Choline acetyl-transferase | ALS, AD, ONND |
| Tyrosine hydroxylase | PD |

Abbreviations:
ALS amyotrophic lateral sclerosis;
AD Alzheimer's disease;
PD Parkinson's disease;
CVA cerebrovascular accident (stroke),
SCI spinal cord injury
ONND other neurodegenerative and neurological diseases (e.g. Huntington's disease)
MS multiple sclerosis
IAP inhibitor of apoptosis proteins
Mammalian IAPs - naip, xiap/hilp/miha, c-lap1/hiap-2/mihb, c-iap2/hiap-1/mihc

The invention utilizes hybrid proteins. According to one aspect, a hybrid protein comprises a therapeutic molecule joined by a covalent bond to TTC, or a portion thereof that retains the central nervous system delivery function described herein. Hybrid proteins containing TTC can be constructed as fusion proteins, e.g., in which TTC is joined to a therapeutic molecule by a peptide bond or by a peptide linker, or as chemical conjugates, in which TTC is connected by a linker molecule (typically a small organic molecule) to a therapeutic molecule. TTC can be placed at the N-terminus or the C-terminus of the fusion protein, and can be conjugated to a therapeutic molecule in any spatial configuration.

The preferred method for obtaining the hybrid protein molecules of this invention is by recombinant production, which involves genetic transformation of a host cell with a recombinant DNA vector encoding a hybrid protein (i.e., a fusion protein), expression of the recombinant DNA in the transformed host cell to make recombinant protein, and collection and purification of the hybrid protein. Preferably, the host organism is unicellular. More preferably, the host organism is prokaryotic. Eukaryotic cells are preferred for expression of the hybrid protein when the protein linked to TTC may be insoluble in bacteria or requires post-translational modification. In such cases, standard eukaryotic cells can be used for production of the hybrid proteins, such as mammalian cells, insect cells and yeast cells. Particularly preferred for preparation of the hybrid proteins are proteins that are secreted from eukaryotic cells, such as by attaching a cleavable leader peptide sequence; many examples of such arrangements are known in the art.

The nucleotide sequence encoding the hybrid protein(s) must be operatively linked to suitable expression control sequences, and is typically incorporated into a plasmid expression vector using conventional recombinant DNA techniques. See generally, Sambrook et al., Molecular Cloning—A Laboratory Manual, Cold Spring Harbor Press (1989); Ausubel et al., (eds.) Current Protocols in Molecular Biology John Wiley & Sons, Inc. (1994). The design and construction of suitable expression vectors for producing the hybrid protein molecules of the invention can be performed by the person of ordinary skill in the art with no more than routine experimentation.

The expression vector is selected to be compatible with the host organism. A wide variety of host/expression vector combinations can be employed for expressing the hybrid protein molecule encoding DNA. Numerous host cell lines, expression vectors, and expression vector components are commercially available. Compatible host/expression vector combinations can be readily selected by those of skill in the art. In one preferred embodiment of the invention, the unicellular host organism is E. coli, and the expression vector is pET28a (Novagen, Madison, Wis). In another preferred embodiment of the invention, the host cells used are insect cells (e.g., High Five™ cells (Invitrogen Catalog no. B855-01), and the expression vector is a vector that facilitates secretion of the hybrid protein (e.g., pMelBac, Catalog no. V1950-20, Invitrogen, Carlsbad, Calif.). (See also Examples section.)

The complete amino acid sequence of tetanus holotoxin and the C. tetani DNA sequence that encodes it have been published (Eisel et al., EMBO J., 5:2495-2502, 1986; Fairweather et al., Nuc. Acids Res., 14:7809-7812, 1986). In addition, the TTC portion of the sequence has been defined, and TTC has been cloned and expressed (Fairweather et al., J. Bacteriol., 165:21-27, 1986; Halpern et al., Infection and Immunity, 58:1004-1009, 1990). A codon-engineered cDNA for TTC has also been constructed to improve expression of this recombinant protein in E. coli (Makoff et al., Nucl. Acids Res. 17:10191-10202, 1989). As used herein, tetanus toxin C fragment also includes molecules having substantially the same neuronal targeting and transport function as TTC, but having some sequence variation (at the nucleotide or amino acid level) relative to the cloned TTC referenced above. Accordingly, a DNA clone encoding the TTC moiety of the hybrid protein of this invention can be obtained by one of ordinary skill in the art, using a publicly available strain of C. tetani (e.g., from the American Type Culture Collection), published sequence information (e.g., Eisel et al., supra; Halpern et al., supra), and conventional recombinant DNA techniques. Additional methods and sources of reagents can be found elsewhere, see, e.g., U.S. Pat. No. 5,965,406 to Murphy and U.S. Pat. No. 5,780,024 to Brown et al., the contents of which are expressly incorporated herein by reference in their entirety.

Therapeutic molecules such as therapeutic proteins also can be conjugated to TTC by non-peptide bonds (i.e. not fusion proteins) to prepare a conjugate capable of delivering the therapeutic molecule. Thus, as used herein “hybrid” molecules includes both fusion proteins and conjugated proteins consisting of TTC and one or more therapeutic molecules, preferably therapeutic proteins. Specific examples of covalent bonds for preparing conjugates include those wherein bifunctional cross-linker molecules are used. The cross-linker molecules may be homobifunctional or heterobifunctional, depending upon the nature of the molecules to be conjugated. Homobifunctional cross-linkers have two identical reactive groups. Heterobifunctional cross-linkers are defined as having two different reactive groups that allow for sequential conjugation reaction. Various types of commercially available cross-linkers are reactive with one or more of the following chemical groups; primary amines, secondary amines, sulfhydryls, carboxyls, carbonyls and carbohydrates. One of ordinary skill in the art will be able to ascertain without undue experimentation the preferred molecule for linking the therapeutic molecule and TTC, based on the chemical properties of the molecules being linked and the preferred characteristics of the bond or bonds.

When delivered via prior methods of peripheral administration (e.g., intravenous, intramuscular), hybrid proteins that include TTC are believed to be internalized by selected types of primary-order neurons, transported retrogradely to the CNS, and perhaps transported transsynaptically to second-order neurons. Therefore, prior art methods provide delivery of TTC only to neurons that initially internalized the hybrid protein and those that have synaptic contact with the first-order neuron. This results in delivery of therapeutic proteins to restricted cell types in restricted areas of the brain, primarily motor neurons in the spinal cord and brainstem. This pattern of distribution is not optimal for treating disorders that affect other regions of the brain.

In contrast, the present invention provides enhanced delivery of therapeutic molecules (preferably but not exclusively proteins) to multiple types of neurons in a wide pattern of distribution throughout the brain and spinal cord. Enhanced delivery in accordance with the invention has two aspects, each of which can be accomplished independently by the invention.

In a first aspect, by “enhanced delivery” is meant that a therapeutic molecule is delivered to types of neurons and regions of the brain and spinal cord that would not be accessible to molecules via retrograde transport in motor neurons that project outside the CNS, or via transsynaptic transport from such motor neurons. With regard to enhanced delivery of a TTC-linked therapeutic protein following intraventricular administration, we believe that TTC may have certain unspecified physicochemical properties that allow it to cross the ependymal cell lining of the brain more efficiently than the therapeutic protein would do in the unmodified form.

It must be emphasized that it is unexpected that TTC can enhance uptake of compounds such as proteins into the brain and spinal cord from the cerebrospinal fluid. This method of enhancing the uptake of exogenous proteins into the CNS might be expected by a person of ordinary skill in the art given that TTC and TTC fusion proteins readily bind to neurons in culture. However, the critical issue in delivery of TTC and TTC fusion proteins from cerebrospinal fluid is that there is an intervening barrier between the cerebrospinal fluid and the brain and spinal cord tissues that prevents TTC from binding directly to neurons. This barrier consists at least in part of meningeal cells located on the outer surface of the CNS as well as ependymal cells that line the cerebral ventricles and central canal of the spinal cord. Indeed, there are precedents documenting that many proteins do not penetrate the brain and spinal cord from the cerebrospinal fluid. For example, studies in which the growth factor BDNF has been chronically infused into the cerebrospinal fluid of the ventricles have shown that BDNF is primarily bound by ventricular ependymal cells with only limited diffusion into the adjacent brain parenchyma (Kordower et al., Exp. Neurol. 124: 21-30, 1993; Anderson et al., J. Comp. Neurol. 357: 296-317, 1995). An important aspect of the invention is that TTC facilitates delivery of itself and its passenger molecules through the ependymal lining of the brain and spinal cord.

An example of regions of the brain not accessible to molecules via retrograde transport or transsynaptic transport are higher order regions of the brain. As used herein, “higher order regions of the brain” means regions of the brain other than the spinal cord or brainstem. Specifically, higher order regions of the brain include areas of the cerebral cortex, hippocampus, thalamus, basal ganglia and hypothalamus as detailed below.

Various areas of the cerebral cortex process sensory information or coordinate motor output necessary for control of movement. These areas that coordinate and process information are divided into primary, secondary and tertiary sensory or motor areas.

The primary sensory areas receive information from peripheral receptors with only a few synapses interposed. Surrounding the primary areas are the higher order secondary and tertiary sensory and motor areas. These areas integrate information coming from the primary sensory areas. In contrast, higher order motor areas send complex information required for a motor act to the primary motor cortex, which has projections to the brainstem and spinal cord.

The cerebral cortex also contains regions called association areas, which lie outside the sensory and motor areas, and make up the largest area of cortex. The association areas are: the parietal-temporal-occipital association cortex, which integrates somatic sensations, hearing and vision inputs to form complex perceptions; the prefrontal association cortex, which is involved in voluntary movements and selected executive and cognitive functions; and the limbic association cortex, which contributes to motivation, emotion and memory.

Additional higher order regions of the brain include the hippocampus, which converts short term memory to more permanent memory, and recalls spatial relationships; the thalamus, which relays information from the brainstem and spinal cord to the cerebral cortex; and the hypothalamus, which controls homeostasis of bodily functions, and contributes to behavioral responses.

In a second aspect, by “enhanced delivery” is meant that a therapeutic molecule is delivered to a greater proportion or volume of the brain and spinal cord than is possible with retrograde and transsynaptic transport. In certain embodiments of the invention, this means that therapeutic molecules are delivered to greater than about 10% of the brain, preferably greater than about 20% of the brain, more preferably greater than about 30% of the brain, yet more preferably greater than about 40% of the brain, still more preferably greater than about 50% of the brain, more preferably greater than about 60% of the brain, even more preferably greater than about 70% of the brain, still more preferably greater than about 80% of the brain and most preferably greater than about 90% of the brain, In the embodiments of the second aspect, the enhanced delivery is to substantially all regions of the brain, particularly to regions of the brain not accessible via retrograde transport or transsynaptic transport, but also to regions of the brain that are accessible via retrograde transport or transsynaptic transport. Similarly enhanced delivery to the spinal cord is made possible using the methods of the invention.

An important embodiment of the invention provides for treatment of subjects with a neurological disorder that affects higher order regions of the brain or non-motor regions of the spinal cord using a TTC hybrid protein. A “neurological disorder” is defined herein as a disorder that affects the central nervous system (including the brain and spinal cord) and/or peripheral and/or autonomic nervous system. Neurological disorders include but are not limited to cerebrovascular accidents (strokes), trauma to the brain and spinal cord, epilepsy, pain syndromes, metabolic and infectious and autoimmune disorders of the central and peripheral nervous system, neurological cancers, developmental nervous system diseases and neurodegenerative disorders. A “neurodegenerative disorder” is defined herein as a condition in which there is progressive loss of neurons in the nervous system. One of ordinary skill in the art would be familiar with the target area in the brain of a subject affected by a stroke, trauma or a neurodegenerative disorder and therefore in need of treatment according to the instant invention. There are diverse sub-types of strokes that can affect either the brain or the spinal cord; similarly, diverse types of trauma can affect these regions as well. Most are by definition acute processes. Examples of neurodegenerative disorders include a broad group of chronic neurodegenerative conditions such as familial and sporadic amyotrophic lateral sclerosis (FALS and ALS, respectively), familial and sporadic Parkinson's disease, Huntington's disease, familial and sporadic Alzheimer's disease, fronto-temporal dementia, multiple sclerosis, spinocerebellar atrophies such as olivopontocerebellar atrophy, multiple system atrophy, progressive supranuclear palsy, diffuse Lewy body disease, corticodentatonigral degeneration, progressive familial myoclonic epilepsy, striatonigral degeneration, torsion dystonia, familial tremor, Down's Syndrome, Gilles de la Tourette syndrome, Hallervorden-Spatz disease, familial and sporadic peripheral neuropathies, dementia pugilistica, AIDS dementia, age-related dementia, age-associated memory impairment, and amyloidosis-related neurodegenerative diseases such as those caused by the prion protein (PrP) which is associated with transmissible spongiform encephalopathies (e.g., Creutzfeldt-Jakob disease, Gerstmann-Straussler-Scheinker syndrome, scrapie, and kuru), and those caused by excess cystatin C accumulation (hereditary cystatin C angiopathy). The present invention is also likely to have application to other conditions in which there is acute or sub-acute deterioration of the brain including not only stroke and trauma, as noted above, but also cerebral edema, surgery-related brain injury, metabolic brain diseases such as Wernicke-Korsakoff's dementia, and acute peripheral nerve injury. These examples are not meant to be comprehensive but serve merely to illustrate potential applications for this invention.

Most of the chronic neurodegenerative diseases are typified by onset during the middle adult years and lead to rapid degeneration of specific subsets of neurons within the nervous system, ultimately resulting in premature death.

Alzheimer's disease is one of the most important of the neurodegenerative diseases due to the high frequency of occurrence within the population and the fatal course of the disease. It is characterized by loss of function and death of nerve cells in several areas of the brain leading to loss of cognitive function such as memory and language. There are both familial and sporadic forms of Alzheimer's disease, and there are both early onset (presenile) and later onset (senile) forms of the disease; in all of these forms, there is considerable overlap in the general pathological findings. The cause of nerve cell death is unknown but the affected neurons are recognized by the presence of unusual helical protein filaments within the cells (neurofibrillary tangles), by extracellular deposition of another abnormal protein, beta amyloid, and by neuronal degeneration. There is also gross atrophy in cortical regions of brain, especially frontal and temporal lobes. A clear genetic predisposition has been found for presenile dementia. Familial autosomal dominant cases have been reported and the majority of individuals with trisomy 21 (Down's syndrome) develop presenile dementia after the age of 40.

Amyotrophic lateral sclerosis (ALS) is the most commonly diagnosed progressive motor neuron disease. The disease is characterized by degeneration of motor neurons in the cortex, brainstem and spinal cord (Harrison's Principles of Internal Medicine, 1991 McGraw-Hill, Inc., New York; Tandan et al. Ann. Neurol, 18:271-280, 419-431, 1985). Generally, the onset is between the third and sixth decade, typically in the sixth decade; ALS is uniformly fatal. Although some genetic bases have been demonstrated (e.g., mutations in superoxide dismutase gene on chromosome 21; see Rosen et al., Nature 362:59-62, 1993), these genetic abnormalities do not uniformly exist in ALS patients, and thus the full spectrum of causes of the disease is yet unknown.

In ALS motor neurons of the cerebral cortex, brainstem and anterior horns of the spinal cord are affected. The class of neurons affected is highly specific: motor neurons for ocular motility and sphincteric motor neurons of the spinal cord remain unaffected until very late in the disease. Death in ALS is generally due to respiratory failure secondary to profound generalized and diaphragmatic weakness. About 10% of ALS cases are inherited as an autosomal dominant trait with high penetrance after the sixth decade (Mulder et al. Neurology, 36:511-517, 1986; Horton et al. Neurology, 26:460464, 1976). In almost all instances, sporadic and autosomal dominant familial ALS (FALS) are clinically similar (Mulder et al. Neurology, 36:511-517, 1986; Swerts et al., Genet. Hum, 24:247-255, 1976; Huisquinet et al., Genet. 18:109-115, 1980). As noted, in some but not all FALS pedigrees the disease is caused by defects in the gene on chromosome 21q that encodes the cytosolic protein, Cu/Zn superoxide dismutase (Rosen et al., Nature 362:59-62, 1993). While a single drug has been approved by the F.D.A. for treatment of ALS, its effect is minimal at best; there is no primary therapy for ALS.

Parkinson's disease (paralysis agitans) is a common neurodegenerative disorder that appears in mid to late life. Familial and sporadic cases occur, although familial cases account for only 1-2 percent of the observed cases. Patients frequently have nerve cell loss with reactive gliosis and formation of Lewy bodies in the substantia nigra and locus coeruleus of the brainstem. Similar changes are observed in the nucleus basalis of Meynert and, in the long term, the nerve cell loss may be quite widespread. As a class, the nigrostriatal dopaminergic neurons seem to be most affected. The disorder generally develops asymmetrically with tremors in one hand or leg and progresses into symmetrical loss of voluntary movement. Eventually, the patient becomes incapacitated by rigidity and tremors. In the advanced stages the disease is frequently accompanied by dementia. Diagnosis of both familial and sporadic cases of Parkinson's disease can only be made after the onset of the disease. While there are symptomatic therapies for Parkinson's disease, there is no primary treatment that slows the underlying neurodegeneration in this disease.

Huntington's disease is a progressive disease characterized by a movement disorder and dementia; it is always transmitted as an autosomal dominant trait. Individuals are asymptomatic until the middle adult years, although some patients show symptoms as early as age 15. Once symptoms appear, the disease is characterized by choreoathetotic movements and progressive dementia until death occurs 15-20 years after the onset of symptoms. Patients with Huntington's disease have progressive atrophy of the caudate nucleus and the structures of the basal ganglia. Atrophy of the caudate nucleus and the putamen is seen microscopically where there is an excessive loss of neural tissue. However, there are no morphologically distinctive cytopathological alterations.

Although some of the characteristic mental depression and motor symptoms associated with Huntington's disease may be suppressed using tricyclic antidepressants and dopamine receptor antagonists, respectively, no therapy exists for slowing or preventing of the underlying disease process. Huntington's disease appears to map to a single gene on chromosome 4 that encodes a protein known as “huntingtin”. The huntingtin gene in its mutant form contains pathological expansions of CAG repeats (see U.S. Pat. No. 5,686,288). A genetic test currently exists for the clinical assessment of disease risk in presymptomatic individuals with afflicted relatives but there is no primary therapy for Huntington's disease.

Hallervorden-Spatz disease is a neurodegenerative disease that affects neurons in the region of the basal ganglia. Symptoms generally first appear during childhood or adolescence and the disease seems to be inherited in an autosomal recessive fashion. Patients show abnormalities in muscle tone and movement such a choreoathetosis and dystonia similar to that seen in parkinsonism. As the disease progresses there is increasing dementia. Death generally occurs approximately ten years after onset. There is no known presymptomatic diagnosis, cure or treatment for Hallervorden-Spatz disease. However, iron toxicity has recently been implicated in the progression of this disease (Greenfield, Neuropathology, W. Blackwood & J. A. N. Corsellis, Eds. (Edinborgh; T. and A. Constable, Ltd., 1976) pages 178-180). As a result of this implication, the chelating agent deferox amine mesylate has been administered to patients. However, this therapeutic approach has shown no definite benefit (Harrison's Principles of Internal Medicine, Wilson et al. Eds., McGraw-Hill, Inc., New York, 1991). There is no primary treatment for this highly debilitating disease.

A large group of neurodegenerative diseases are described as the “spinocerebellar atrophies” (SCA). These all entail progressive degeneration of various subsets of neurons in the cerebellum, brainstem and other regions of the neuraxis; most are inherited as dominant traits. Among these are the olivopontocerebellar atrophies (OPCA), which include a number of disorders characterized by a combination of cerebellar cortical degeneration, atrophy of the inferior olivary nuclei, and degeneration of the pontine nuclei in the basis pontis and middle cerebellar peduncles. Many of the spinocerebellar atrophies arise from expansions of CAG repeat domains in different types of neurons (for example, Orr et al., Nature Genetics 4:221-226, 1993).

The hybrid protein molecules of the invention are administered in effective amounts. An effective amount is a dosage of the hybrid protein molecule sufficient to provide a medically desirable result. The effective amount will vary with the type of therapeutic protein coupled to TTC, the particular condition being treated, the age and physical condition of the subject being treated, the severity of the condition, the duration of the treatment, the nature of the concurrent therapy (if any), the specific route of administration and like factors within the knowledge and expertise of the health practitioner. For example, in connection with a neurological disorder in a subject, an effective amount is that amount which inhibits or reduces the severity of symptoms of the neurological disorder. Thus, it will be understood that the hybrid protein molecules of the invention can be used to treat the above-noted conditions according to the preferred modes of administration. It is preferred generally that a maximum dose be used, that is, the highest safely tolerated dose according to sound medical judgment.

A subject, as used herein, refers to any mammal (preferably a human, and including a non-human primate, cow, horse, pig, sheep, goat, dog, cat or rodent) with a condition requiring delivery to regions of the brain and spinal cord not accessible via retrograde transport or transsynaptic transport from the periphery.

A hybrid protein molecule of the invention may be administered alone or as part of a pharmaceutical composition. Such a pharmaceutical composition may include the hybrid protein molecule in combination with any standard physiologically and/or pharmaceutically acceptable carriers which are known in the art. The compositions should be sterile and contain a therapeutically effective amount of the hybrid protein molecule in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material (to cells other than neuronal cells) that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Vehicles for administration of the hybrid proteins include but are not limited to sodium chloride solution, Ringer's dextrose, dextrose and sodium chloride, lactated Ringer's or fixed oils.

Compositions suitable for parenteral administration conveniently comprise sterile aqueous and non-aqueous preparations of the hybrid protein molecules of the invention. This aqueous preparation may be formulated according to known methods using suitable dispersing or wetting agents and suspending agents. Aqueous carriers include water, alcoholic/aqueous solutions, emulsions or suspensions, including saline and buffered media. Examples of non-aqueous solvents are propylene glycol, polyethylene glycol, vegetable oils such as olive oil, and injectable organic esters such as ethyl oleate, and including synthetic mono- or di-glycerides. The sterile injectable or infusable preparation also may be a sterile injectable or infusable solution or suspension in a non-toxic parenterally-acceptable diluent or solvent, for example, as a solution in 1,3-butane diol. In addition, fatty acids such as oleic acid may be used in the preparation of injectables or infusables. Carrier formulations suitable for various routes of administration can be found in Remington's Pharmaceutical Sciences, Mack Publishing Co., Easton, Pa. Preservatives and other additives may also be present such as, for example, antimicrobials, anti-oxidants, chelating agents, and inert gases and the like.

For increased delivery of therapeutics to multiple regions of the brain and spinal cord using the hybrid TTC proteins as disclosed herein, two distinct preferred modes of administration are available. The particular mode selected will depend, of course, upon the particular drug selected, the severity of the condition being treated, and the dosage required for therapeutic efficacy. The methods of the invention, generally speaking, may be practiced using any mode of administration that is medically acceptable, meaning any mode that produces effective levels of the active compounds without causing clinically unacceptable adverse effects.

One mode of administration is direct administration into the brain, such as by injection, infusion or implantation of a sustained release formulation. A second mode of administration is introduction of the hybrid proteins into the cerebrospinal fluid by infusion. This mode of administration includes any method for delivering the hybrid proteins into the cerebrospinal fluid, such as via the subarachnoid space of the spinal cord (intrathecal) orvia the cerebral ventricles (intracerebroventricular, icv).

According to preferred embodiments, the hybrid TTC proteins are administered continuously for a prolonged period of time via infusion or sustained release formulations to provide effective delivery of the therapeutic compounds to the higher order regions of the brain and spinal cord. Infusion of the hybrid proteins of the invention is for 1 or more days, preferably 3 or more days, and more preferably 1 or more weeks. In certain embodiments of direct administration into the brain and administration to the cerebrospinal fluid, a pump device is used to administer substantial amounts of the hybrid proteins continuously for a period of days, weeks, months or years.

An alternative mode of administration useful in any of the methods of the invention is intranasal administration. In this mode, the hybrid TTC proteins are administered via any conventional intranasal delivery device, and are taken up by olfactory sensory neurons that project to the brain.

The pharmaceutical compositions may conveniently be presented in unit dosage form and may be prepared by any of the methods well-known in the art of pharmacy. All methods include the step of bringing the hybrid protein molecules of the invention into association with a carrier which constitutes one or more accessory ingredients. In general, the compositions are prepared by uniformly and intimately bringing the hybrid protein molecules into association with a liquid carrier, a finely divided solid carrier, or both, and then, if necessary, shaping the product.

Compositions suitable for administration by direct administration into the brain or infusion into the cerebrospinal fluid include suspensions in aqueous liquids or non-aqueous liquids such as a syrup, elixir or an emulsion.

Thus the invention provides dosage forms appropriate to the methods of administration of the TTC hybrid proteins. For infusion, pump-based hardware delivery systems can be used, some of which are adapted for implantation. For example, for infusion a medical device is provided that includes a reservoir for TTC hybrid proteins and a mechanism for substantially continuous delivery of the TTC hybrid proteins, such as a pump and appropriate fluid handling apparatus. For intranasal delivery, a suitable intranasal pharmaceutical delivery device containing a TTC hybrid protein of the invention and other standard carriers, propellants and the like is provided.

Other delivery systems can include time-release, delayed release or sustained release delivery systems. Such systems can avoid repeated administrations of the hybrid protein molecules of the invention, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the hybrid protein molecule is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686.

Use of a long-term sustained release implant may be particularly suitable for treatment of chronic conditions. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

The hybrid protein molecules of the invention may be administered alone or in combination (co-administered) with other drug therapies. The selection of other drug therapies (e.g., as additional active ingredients in a pharmaceutical formulation) will depend on considerations such as the particular neurological disease(s) or disorder(s) for which the composition is to be used, and potential adverse drug interactions. The term “co-administered,” means administered substantially simultaneously with another agent. By substantially simultaneously, it is meant that a hybrid protein molecule of the invention is administered to the subject close enough in time with the administration of the other agent. The other agent may be present in a different formulation than the hybrid protein molecule of the invention, or it may be part of the same formulation.

The co-administered agent can act cooperatively, additively or synergistically with a hybrid protein molecule of the invention to produce a desired effect, for example, lessening of neurodegeneration or symptoms thereof. The other agent is administered in effective amounts. Such amounts maybe less than these sufficient to provide a therapeutic benefit when the agent is administered alone and not in combination with a hybrid protein molecule of the invention. A person of ordinary skill in the art would be able to determine the effective amounts needed.

Co-administered agents include: for Huntington's disease: coenzyme Q10, remacemide, SCH 58261, ethyl-ester of eicosapentaenoic acid (ethyl-EPA), olanzapine, pramipexole, levodopa, donepezil, physostigmine, tacrine; for Alzheimer's disease: tacrine, galantamine, donepezil, rivastigmine, anti-inflammatory drugs; Parkinson's disease: rasagiline, tolcapone, pramipexole, levodopa, budipine; for amyotrophic lateral sclerosis: minocycline, riluzole, topiramate.

The invention will be more fully understood by reference to the following examples. These examples, however, are merely intended to illustrate the embodiments of the invention and are not to be construed to limit the scope of the invention.

### EXAMPLE

### Example 1

### Intraventricular Infusions of TTC:SOD Fusion Protein

The recombinant SOD:TTC fusion protein used in the examples described below was expressed in E. coli from a fusion gene assembled in the commercial plasmid expression vector, pET28a (Novagen). The new SOD:TTC fusion gene used the same modified cDNA for human SOD-1 employed in our first construct (see Francis et al., J. Biol. Chem. 270:15434-15442, 1995). However, the new fusion gene construct was different from the previous construct in two important regards. First, the TTC cDNA used in the new SOD:TTC fusion gene is a codon-engineered cDNA construct having substantial changes in deoxyribonucleotide sequence compared to the wild-type cDNA sequence for TTC as encoded by Clostridium tetani. This modified TTC cDNA of Makoff et al. (Nucl. Acids Res. 17:10191-10202, 1989) has had many of the rare codons for isoleucine, glycine, and arginine present in the native sequence replaced with codons that are more commonly used by E. coli. The new sequence ultimately permitted a substantial increase in the amount of soluble recombinant TTC protein expressed in bacteria. Second, our use of a different prokaryotic expression vector, pET28a, allowed us to add a polyhistidine tag at the amino terminus of the SOD:TTC fusion protein. The polyhistidine affinity tag was engineered into our new fusion protein to allow us to more efficiently obtain the large quantities of SOD:TTC necessary for the intracerebroventricular infusion study in rats.

The pET28a:SOD:TTC plasmid was overexpressed in E. coli strain BL21(Star)DE3 (Invitrogen, Carlsbad, Calif.) using the induction parameters previously described in Francis et al (J. Neurochem. 74:2528-2536, 2000). The SOD:TTC target protein was subsequently purified from total soluble bacterial protein using 40% ammonium sulfate precipitation followed by immobilized metal affinity chromatography IMAC). IMAC was performed under nondenaturing conditions using Nickel NTA resin (Qiagen, Valencia, Calif.) according to the manufacturers instructions.

For the infusion studies, female rats (300-325 g) were anesthetized and then implanted with a unilateral cannula in the left cerebral ventricle. The cannula was connected by polyethylene tubing to a 2 ml Alzet osmotic minipump (10 μl/hour; Durect Corporation, Cupertino, Calif.), which was then implanted subcutaneously. Rats were continuously infused for three days with either phosphate-buffered saline (PBS), human superoxide dismutase (hSOD-1; 1 mg/ml), tetanus toxin fragment C (TTC; 3 mg/ml), or SOD:TTC fusion protein (4 mg/ml). In light of both the high cost of commercial recombinant TTC and the relatively large quantities of TTC required for chronic infusion vs. acute intraparenchymal injection, the recombinant 6× His:TTC used in this i.c.v. infusion study was produced and purified in our own laboratory. All protein samples were extensively dialyzed against PBS before sterilization with a 0.22 μm syringe filter.

At the end of the three day infusion period, the rats were euthanized and the mini-pumps quickly removed for subsequent determination of residual sample volume. Animals were then fixed by perfusion with 4% formaldehyde/PBS prior to removal of whole brains and spinal cords. After overnight postfixation, tissues were sectioned using an EMS-4000 Oscillating Tissue Slicer (Electron Microscopy Sciences, Fort Washington, Pa.). Tissue sections were then processed for anti-TTC or anti-human SOD-1 immunoreactivity as previously described (Francis, et al., Exp. Neurol. 146: 435-443, 1997).

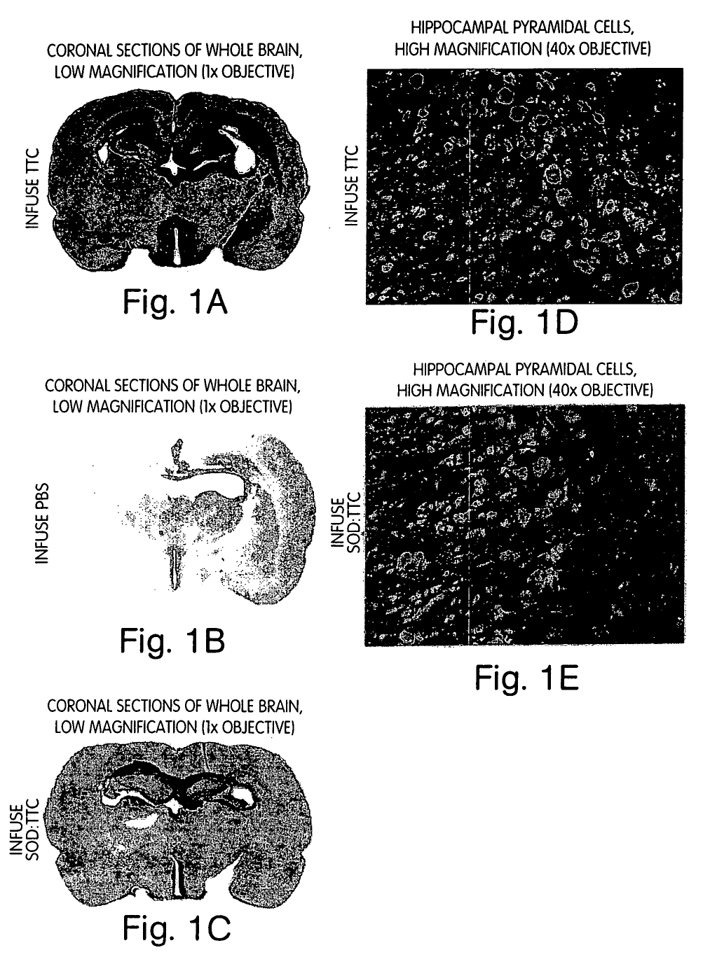

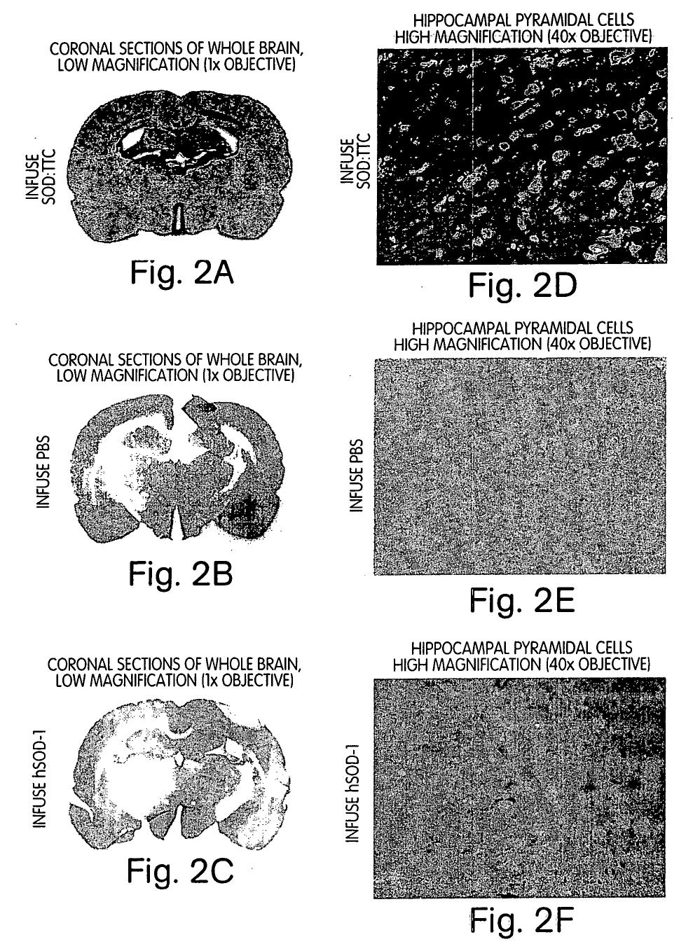

Brain sections from one of the rats infused intraventricularly with TTC showed moderate to strong anti-TTC immunostaining located diffusely in all the major regions of gray matter present in the section (e.g. hippocampus, cerebral cortex, thalamus, and hypothalamus) (FIG. 1A). While the strongest labeling was invariably found in areas of gray matter closest to the ventricles, substantial levels of signal were present in all the remaining regions of gray matter examined. The lower levels of horseradish peroxidase reaction product seen throughout the rest of the brain appeared specific for TTC given the virtual absence of anti-TTC immunoreactivity observed in brain sections from rats treated with PBS (FIG. 1B). Brain sections from animals treated by intraventricular infusion of SOD:TTC similarly showed increased levels of both anti-TTC (FIG. 1C) and anti-SOD-1 (FIG. 2A) and immunoreactivity throughout the brain, although the intensity and distribution of signal in the SOD:TTC sections was less than that observed in the sections from animals treated with TTC. By contrast, at this level of the brain, sections from rats infused with either PBS (FIG. 2B) or hSOD-1 (FIG. 2C) revealed only modest levels of anti-hSOD-1 immunoreactivity. Anti-hSOD-1 staining in sections from animals treated with hSOD-1 was nonetheless notably greater than that seen in sections of PBS-treated animals. With the clear exception of sections obtained from PBS-treated animals, most brain sections in the SOD:TTC and TTC infused animals revealed that the intensity and distribution of anti-TTC or anti-SOD-1 signal was generally greater in one side of the brain as compared to the other. This finding was probably attributable to the ipsilateral location of the infusion cannula.

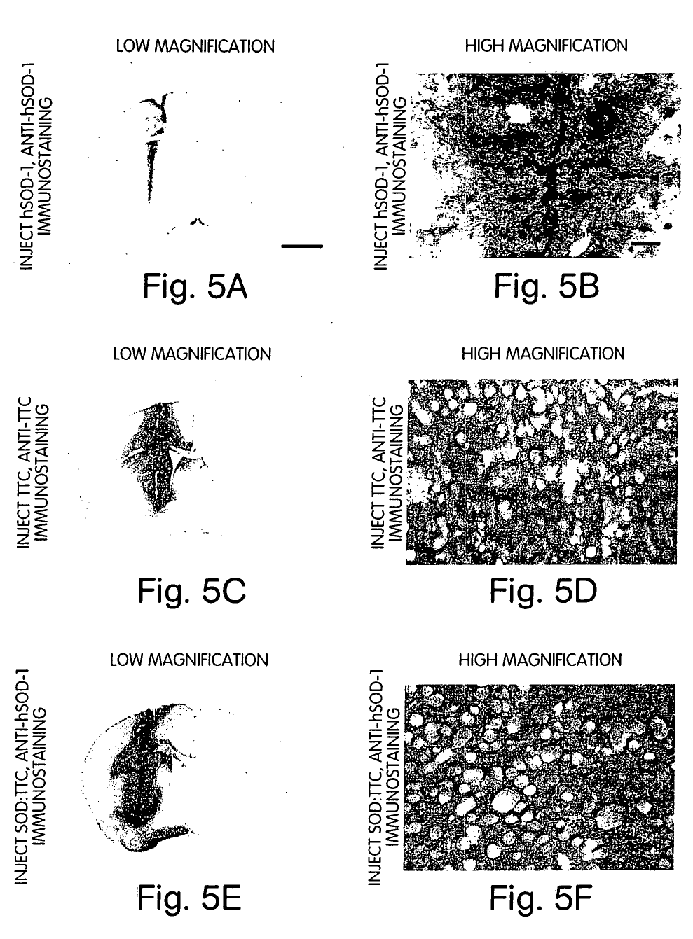

At higher magnification, examination of sections from either TTC- or SOD:TTC-infused animals (FIGS. 1D, 1E, 2D) generally revealed a pattern of staining essentially identical to that seen in the striata of mice that had received intraparenchymal injections of either TTC or SOD:TTC (see Example 2, FIG. 5, below). Thus, much of the immunolabeling appeared to be located in the neuropil and extracellular space. However, further inspection of hippocampal pyramidal cells in sections from rats treated by intraventricular infusion with either TTC (FIG. 1D) or SOD:TTC (FIG. 1E and FIG. 2D) additionally revealed moderate levels of perinuclear staining within cell bodies and strong staining of apical dendrites (denoted by darkly stained wavy lines in the left side of FIGS. 1D and 1E and in FIG. 2D). Similar examination of hippocampal pyramidal neurons in sections from animals infused with either PBS or hSOD-1 (FIGS. 2E and 2F, respectively) showed no definitive evidence of any cell-associated immunoreactivity.

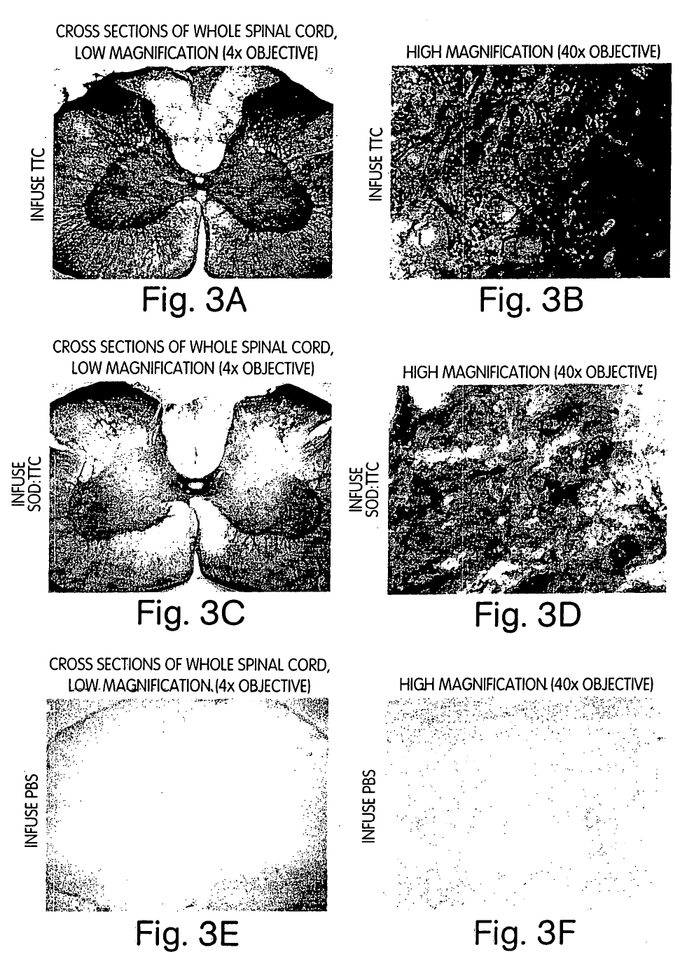

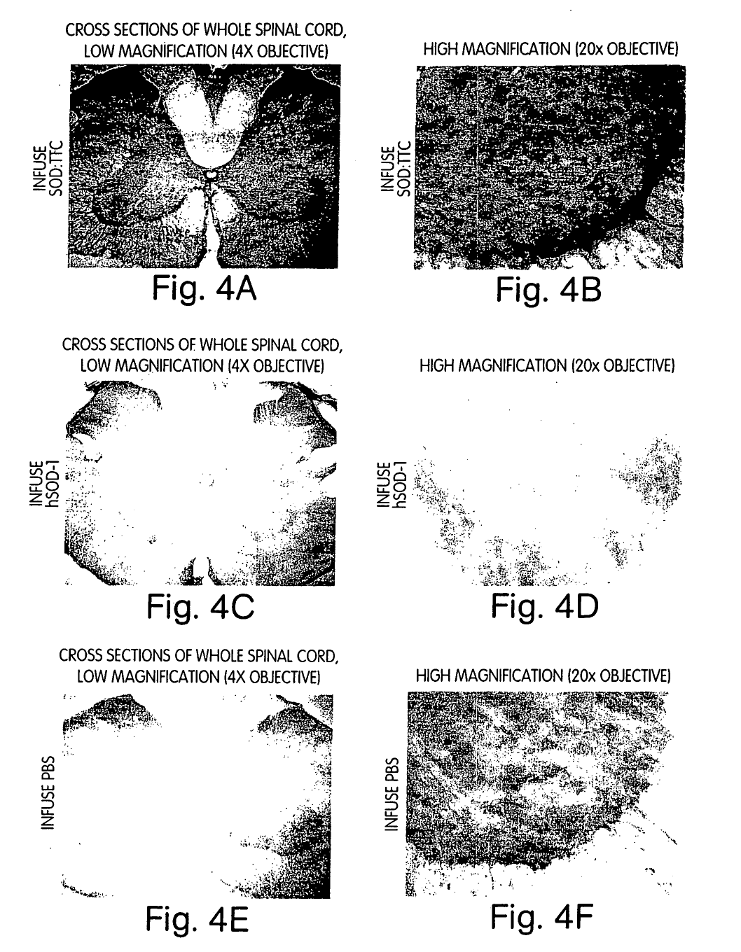

Consistent with the results in the brain, spinal cord sections obtained from the TTC-infused animal described above showed intense anti-TTC staining concentrated in the gray matter of both the ventral and dorsal horns (FIG. 3A). In particular, a major part of the anti-TTC immunostaining in the ventral horn appeared to be associated with the cell bodies and proximal dentrites of large motor neurons (FIG. 3B). Strongly-immunoreactive punctate structures were seen to dot the entire surface of several motor neuron cell bodies. We also observed in some cells the presence of labeled, intracellular granules often found in a perinuclear location. Spinal cord sections from rats intraventricularly infused with SOD:TTC similarly showed strong anti-TTC immunostaining in the ventral horn that was predominantly associated with motor neurons (FIGS. 3C, 3D). Anti-TTC immunoreactivity in spinal cord sections of TTC- and SOD:TTC-treated rats was specific for TTC given that sections from animals infused with PBS showed no horseradish peroxidase reaction product (FIGS. 3E, 3F). Spinal cord sections from SOD:TTC-treated animals also showed strong immunolabeling of the ventral horn with anti-SOD-1 antibody although staining in motor neurons appeared less than that seen with the anti-TTC antibody (FIGS. 4A, 4B). Spinal cord sections from animals infused with hSOD-1 (FIG. 4C, 4D) showed a uniform pattern of weak anti-SOD-1 immunostaining in the gray matter that was essentially identical to that seen in cord sections from animals treated with PBS (FIGS. 4E, 4F).

### Example 2

### Intracerebral Injections of TTC:SOD Fusion Protein

Under pentobarbital anesthesia, adult male mice (C57BL6 strain) underwent limited craniotomy using aseptic techniques. Intracerebral injections of PBS or test proteins into the left striatum were performed by stereotactic localization using a 28G Hamilton syringe. Three microliters of each test reagent was injected under manual control. Recombinant tetanus toxin C-fragment (TTC, catalog no. 1348655, Roche Applied Science, Indianapolis, Ind.) and SOD:TTC fusion protein were administered at a concentration of 5 mg/ml while human SOD-1 (Sigma Chemical Co., St. Louis, Mo.) was given at a concentration of 1.25 mg/ml. The lower concentration of SOD-1 was chosen to maintain molar equivalence to SOD:TTC. To avoid solution reflux, the needle was left in position for an additional 3 minutes after the injection was completed.

Animals were allowed to survive for 48 hours postinjection, when they were euthanized by pentobarbital overdose and perfused with 4% formaldehyde/PBS. Whole brains were removed and postfixed overnight at 4° C. in the same fixative. Tissues were sectioned using an EMS-4000 Oscillating Tissue Slicer. Tissue sections were then processed for anti-TTC or anti-human SOD-1 immunoreactivity as previously described (Francis, et al., Exp. Neurol. 146: 435-443, 1997).

Brain sections from mice that received intrastriatal injections of hSOD-1 showed moderate anti-hSOD-1 immunostaining confined to the margins of the needle tract (FIG. 5A). Examination of this immunoreactivity at higher magnification revealed no definitive pattern of cellular localization (FIG. 5B). In contrast, sections from animals injected with either TTC (FIG. 5C) or SOD:TTC (FIG. 5E) demonstrated strong immunolabeling with anti-TTC and anti-SOD-1 antibodies, respectively, that extended for a considerable distance into the adjacent striatal and cortical tissue. Viewing these sections at higher power showed a similar pattern of immunoreactivity present in both the TTC (FIG. 5D) and SOD:TTC (FIG. 5F) specimens. In particular, most of the horseradish peroxidase reaction product appeared to be present within the extracellular space/neuropil surrounding neuronal cell bodies.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for administering a therapeutic molecule to a subject, comprising:
providing a hybrid protein comprising the therapeutic molecule and tetanus toxin fragment C, and
administering the hybrid protein by infusion of the hybrid protein into the cerebrospinal fluid, or directly into the brain or spinal cord parenchyma.

2. The method of claim 1, wherein the therapeutic molecule is a protein or peptide, a nucleic acid molecule, a virus, an antibody or fragment thereof, a lipid, a polysaccharide, an oligonucleotide or a modified or derivatized oligonucleotide, an RNA molecule or a modified or derivatized oligoribonucleotide, a plasmid, cosmid, bacmid or vehicle for the packaging and/or expression of clonal DNA, or a ribozyme.

3. The method of claim 2, wherein the protein is selected from the group consisting of GDNF, BDNF, LIF, cardiotrophin (CT-1), FGF, HGF, insulin-like growth factors 1 and 2 (IGF-1, IGF-2) and the neurotrophins.

4-12. (canceled)

13. The method of claim 1, wherein the mode of administration is intracerebroventricular administration or intrathecal infusion.

14-18. (canceled)

19. The method of claim 1, wherein the hybrid protein is administered to at least about 10% of brain volume.

20-33. (canceled)

34. The method of claim 1, wherein the hybrid protein is administered directly into the brain or spinal cord parenchyma by injection or infusion.

35-42. (canceled)

43. A method for administering a therapeutic molecule to a region of a subject's brain and spinal cord that is not accessible via retrograde transport or transsynaptic transport from motor neurons, comprising:
providing a hybrid protein comprising the therapeutic molecule and tetanus toxin fragment C, and
administering the hybrid protein by infusion of the hybrid protein into the cerebrospinal fluid, or directly into the brain or spinal cord parenchyma.

44. The method of claim 43, wherein the therapeutic molecule is a protein or peptide, a nucleic acid molecule, a virus, an antibody or fragment thereof, a lipid, a polysaccharide, an oligonucleotide or a modified or derivatized oligonucleotide, an RNA molecule or a modified or derivatized oligoribonucleotide, a plasmid, cosmid, bacmid or vehicle for the packaging and/or expression of clonal DNA, or a ribozyme.

45. The method of claim 43, wherein the protein is selected from the group consisting of GDNF, BDNF, LIF, cardiotrophin (CT-1), FGF, HGF, insulin-like growth factors 1 and 2 (IGF-1, IGF-2) and the neurotrophins.

46-54. (canceled)

55. The method of claim 43, wherein the mode of administration is intracerebroventricular administration or intrathecal infusion.

56-60. (canceled)

61. The method of claim 43, wherein the hybrid protein is administered to at least about 10% of brain volume.

62-75. (canceled)

76. The method of claim 43, wherein the hybrid protein is administered directly into the brain or spinal cord parenchyma by injection or infusion.

77-84. (canceled)

85. A method for treating a neurological disorder, comprising:
administering to a subject in need of such treatment an effective amount of a hybrid protein comprising tetanus toxin fragment C and a therapeutic molecule by infusion of the hybrid protein into the cerebrospinal fluid, or directly into the brain or spinal cord parenchyma.

86. The method of claim 85, wherein the therapeutic molecule is a protein or peptide, a nucleic acid molecule, a virus, an antibody or fragment thereof, a lipid, a polysaccharide, an oligonucleotide or a modified or derivatized oligonucleotide, an RNA molecule or a modified or derivatized oligoribonucleotide, a plasmid, cosmid, bacmid or vehicle for the packaging and/or expression of clonal DNA, or a ribozyme.

87. The method of claim 85, wherein the protein is selected from the group consisting of GDNF, BDNF, LIF, cardiotrophin (CT-1), FGF, HGF, insulin-like growth factors 1 and 2 (IGF-1, IGF-2) and the neurotrophins.

88-96. (canceled)

97. The method of claim 85, wherein the mode of administration is intracerebroventricular administration or intrathecal infusion.

98-102. (canceled)

103. The method of claim 85, wherein the hybrid protein is administered to at least about 10% of brain volume.

104-105. (canceled)

106. The method of claim 85, wherein the subject has a neurological disorder selected from the group consisting of cerebrovascular accidents (stroke), amyotrophic lateral sclerosis, Parkinson's disease, Huntington's disease, Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, striatonigral degeneration, torsion dystonia, familial tremor, Down's Syndrome, Gilles de la Tourette syndrome, Hallervorden Spatz disease, peripheral neuropathies, dementia pugilistica, AIDS dementia, age-elated dementia, age-associated memory impairment, amyloidosis related neurodegenerative diseases, traumatic brain and spinal cord injury, cerebral edema, schizophrenia, peripheral nerve damage, spinal cord injury, and Wernicke Korsakoff's related dementia.

107-118. (canceled)

119. The method of claim 85, wherein the hybrid protein is administered directly into the brain or spinal cord parenchyma by injection or infusion.

120-128. (canceled)
